(19) United States
(12) Patent Application Publication
(10) Pub. No.: US 2024/0082543 A1
(43) Pub. Date: Mar. 14, 2024

(54) Guide Element for a Controllable Vessel Expansion System, and Controllable Vessel Expansion System

(71) Applicant: Xenios AG, Heilbronn (DE)

(72) Inventors: Sven Filipon, Heilbronn (DE); Patrick Rauh, Talheim (DE)

(21) Appl. No.: 17/768,309

(22) PCT Filed: Oct. 16, 2020
(86) PCT No.: PCT/EP2020/079195

§ 371 Date: Apr. 12, 2022

(30) Foreign Application Priority Data
Oct. 17, 2019 (DE) 10 2019 007 222.7

(51) Int. Cl. A61M 25/01

(52) U.S. Cl. CPC A61M 25/0133

(57) ABSTRACT

The present disclosure relates to devices and methods for expanding the tissue of a patient, e.g., guiding members for controllable vessel expansion systems and corresponding controllable vessel expansion systems as well as corresponding methods and applications. Accordingly, a guiding member is suggested for a controllable vessel expansion system, which includes an elongate body couplable to a controllable member of the vessel expansion system and is aligned along the longitudinal axis of the vessel expansion system from a proximal region to a distal region of the vessel expansion system in the coupled state. The body is expandable in a radial direction. Furthermore, the body may be releasably coupled to the controllable member, which together with the guiding member defines a radial extension of the vessel expansion system.

### CROSS-REFERENCE TO RELATED APPLICATIONS

The present application is the national stage entry of International Patent Application No. PCT/EP2020/079195, filed on Oct. 16, 2020, and claims priority to Application No. 10 2019 007 222.7, filed in the Federal Republic of Germany on Oct. 17, 2019, the disclosures of which are expressly incorporated herein in their entireties by reference thereto.

### TECHNICAL FIELD

The present disclosure relates to guiding members for controllable vessel expansion systems as well as vessel expansion systems for insertion into an anatomical region of a patient and corresponding methods.

### BACKGROUND

In a medical application, extension or expansion of tissue in an anatomical region of a patient may be required for various diagnostic and/or therapeutic purposes. For example, a cannula, which may serve as patient access, may be inserted into the patient to monitor a physiological condition and to administer or remove fluids. For example, such a cannula may be inserted into the patient's tissue or a vessel to provide an access to the desired anatomical region. For certain heart diseases and related therapies, a cannula may be inserted into the right atrium via the femoral vein or jugular vein, for example, to relieve the right heart, or it may be provided that the cannula is inserted into the septum to allow transseptal cannulation of the left atrium.

However, such therapies require very precise and non-linear cannula guidance via anatomical curves, which cannot be achieved with standard procedures.

The Seldinger technique is usually used to insert the cannula, so that the access is initially extended to allow the cannula to be inserted, e.g., into a vessel. In general, an incision and/or a puncture with a puncture needle is made in the patient's tissue at the point where the cannula is to be inserted. A Seldinger wire is then inserted through the needle into the opening provided by the incision or puncture and is inserted into a blood vessel, for example, whereupon the needle is removed. In such cases the Seldinger wire serves as a guide wire over which a dilator with a given diameter may be slid. In order to increase the diameter and to allow an expansion of the opening, dilators of different diameters and shapes are successively slid over the Seldinger wire until the desired opening expansion required for the cannula is achieved. The cannula or a catheter may then be pushed over the dilator and both may be inserted together into the blood vessel. Afterwards both the dilator and the Seldinger wire may be removed.

A disadvantage of such an approach is that the achieved expansion is limited to the diameters of the respective dilators. Since the dilators have a given, rigid diameter, several introduction processes are necessary. On the other hand, the achieved diameter is not sufficiently variable, because only a limited set of dilators of different diameters is available. The repeated insertion and removal of the various dilators is therefore not only cumbersome, but may also cause accidental damage to the vascular structure, which may lead to complications. Furthermore, due to their individual rigid structure, the dilators cannot be adapted satisfactorily to the respective vessel or anatomical region on a regular basis, which may be particularly disadvantageous in the case of complex anatomical regions or geometries. In other words, this procedure may not only be time-consuming, but may also be medically disadvantageous and therefore generally forms a burden for the patient concerned.

A further disadvantage of such dilators is that they are designed to be brought into direct contact with the patient's tissue or blood, so that the dilators would have to be sterilely cleaned to comply with hygienic standards and are therefore usually designed as disposable articles for practicality. Dilators may have further components in their inner body, which cannot be separated from the dilator, so that a configuration as a disposable article is hardly justifiable, not only from an economic point of view, but also ecologically.

Accordingly, there is a need to improve the expansion of tissue at the point of entry or opening in such a way that the basic components of a dilator are suitable for multiple uses and facilitate the expansion, and may be configured to be highly variable according to the needs of the individual case.

### SUMMARY

It is an aspect of the present disclosure to enable an improved expansion of tissue by means of a vessel expansion system and to enable a method which controls the cannula to be inserted or may guide or steer it around vascular branches and may control the position of the cannula.

Accordingly, a guiding member is suggested for a controllable vessel expansion system including an elongate body coupleable to a controllable member of the vessel expansion system. In the coupled state, the body is aligned from a proximal region to a distal region of the vessel expansion system along the longitudinal axis of the vessel expansion system. The body is expandable in a radial direction and releasably coupleable to the controllable member, which together with the guiding member defines a radial extension of the vessel expansion system.

The guiding member may serve as an adapter for the controllable member and may therefore be coupled to it to provide a secure attachment to the controllable member for use of a vessel expansion system. The controllable member serves to control or navigate or steer the vessel expansion system and at the same time supports the coupled guiding member during insertion into the tissue of a patient. In other words, the guiding member together with the controllable member may form a unit that may be inserted as such into the tissue of a patient. The controllable member can be at least partially flexible, so that the unit may be adapted in its function as a vessel expansion system to the shape of the respective anatomical region.

In contrast to the known “Seldinger technique”, no separate Seldinger wire is hence required for insertion using systems described herein. Rather, the guiding member and a controllable member together form a vessel expansion system as a functional unit, so that repeated insertion of dilators of different diameters or shapes via a Seldinger wire may be omitted. Furthermore, the diameter in the coupled state is defined both by the body or the guiding member as well as by the controllable member, e.g., since the body and the controllable member in the coupled state can be adjacent to each other in cross-section. The diameter may be adjusted by the radially expandable body and based on a change in the controllable member so that the guiding member may be introduced in the coupled state with a small diameter of the vessel expansion system and the diameter may be variably increased in the inserted state.

Accordingly, the systems described herein may avoid further surgical or rigid elements, which may typically be necessary in the state of the art to support the insertion of the dilator. The guiding member and also a coupled controllable member are therefore decoupled from a guide wire, such as a Seldinger wire. The introduction of a vessel expansion system is significantly simplified, as tissue expansion may take place in a single operation. The burden and also the risk for the patient is hence considerably reduced.

Due to the detachable coupling with a controllable member, the guiding member may furthermore be easily separated, i.e., there is no material bonding connection, so that the controllable member may be manufactured and/or cleaned independently of the guiding member.

In an embodiment, the body may be adapted to be surrounded circumferentially by the controllable member in the coupled state. In other words, in the coupled state, the guiding member may thus be located inside a vessel expansion system and form a functional “core” of the vessel expansion system, wherein the controllable member surrounds the core. Such an arrangement may strengthen the internal structure of a vessel expansion system and may prevent, for example, the external controllable member from being extended or pressed inwards when it is inserted into the tissue or the patient's tissue from accumulating inside the vessel expansion system, thus forming a barrier against further insertion, e.g., into a vessel or into a hollow organ.

In another embodiment, the body is designed as a sleeve or sheath and is adapted to completely enclose the controllable member in the coupled state in a fluid-tight manner. In this case, the guiding member forms an outer layer of the vessel expansion system. It may easily be coupled to or slid over the exterior of a controllable member and serve as an adapter. In this alternative embodiment, the controllable member thus forms a functional “core” of the vessel expansion system. The guiding member prevents fluids from entering the interior of the vessel expansion system, so that the controllable member is sealed against fluids. This configuration avoids potential contamination of the controllable member when using the vessel expansion system, so that the controllable member may be used for further applications without the need for sterile cleaning.

The body can be movable between a first radial extension, wherein the vessel expansion system includes a minimum diameter, and a second radial extension, wherein the body defines a predefined diameter of the vessel expansion system in each radial extension.

Thus an essentially homogeneous outer shape is formed. The diameter or volume may be controlled and adjusted for each radial extension. The guiding member may accordingly form an outer sheath, wherein a change in a coupled controllable member causes a change in the circumference of the outer sheath. There are no wrinkles on the outer surface—regardless of the radial extension. In other words, the guiding member can be preloaded at each extension, so that it may have a substantially convex shape.

The controllable and predefined diameter allows the expansion of a vessel expansion system to be individually adjusted so that the requirements of a medical intervention and/or the anatomy of the patient may be mapped.

In some implementations, the body is furthermore continuously expandable in a radial direction. Thereby, a vessel expansion system in the coupled state of the guiding member may be adapted, for example, to a diameter of a patient's vessel. The tissue or vessel may be successively expanded to achieve sufficient expansion without the need to use dilators of different configuration. Furthermore, the diameter may be specifically adapted to an inner diameter of a cannula or catheter to be inserted, so that—in contrast to the state of the art—an unnecessary expansion of the tissue is not induced, i.e., the expansion does not have to be selected larger than required for the patient due to the rigid, predetermined diameters of conventional dilators.

Stepless or continuous expansion also prevents tissue from being enclosed or pinched, both during expansion and while reducing the vessel expansion system diameter. Also, due to the continuously variable adjustability of the diameter by adjusting the controllable member, the diameter may easily be reduced again so that a vessel expansion system may be removed after insertion of a cannula without affecting the positioning of the cannula.

To effect expansion of the guiding member, the body may include a flexible section in a circumferential direction which defines a radial extension of the body. For example, a portion of the circumference may be stretchable so that a change in the controllable member causes the flexible section to stretch, causing the guiding member to expand at the circumference accordingly.

The body can be made of an elastic material. This allows the flexible section to extend essentially over the entire body and a uniform or homogeneous expansion may be achieved. This also eliminates the need for material bonding or other fixations to a flexible section, thereby improving the structural stability of the guiding member.

Wrinkles on the outer surface of the guiding member may have an undesirable effect on the patient's vessels and a predefined diameter cannot be sufficiently specifically defined in the case of a wrinkle structure on the surface. In some implementations, the body may have a spiral shape in cross-section, whereby the degree of unfolding of the spiral shape defines a radial extension of the body. Thus, a bellows structure or foldable design of the guiding member or flexible section may be avoided and a uniform expansion may be achieved, wherein the expansion is not or not only defined by an elastic property of the guiding member, but may be defined by a continuously rolling or unfolding spiral-shaped flexible section.

To further improve insertion into the tissue, the guiding member may include a friction-reducing coating on an outer surface of the body. For example, a surface tension may be adjusted by the coating so that the guiding member may be more easily inserted into the tissue. Furthermore, when such a guiding member is arranged on the outside, an outer surface of the body may be adapted to discharge or transport fluid, wherein the body includes grooves, lamellae, and/or a honeycomb structure. This prevents fluids from accumulating, while at the same time this avoids a pressure level when inserting a vessel expansion system, thereby facilitating the insertion.

Also, an outer surface of the body may be coated with a drug or medicament, e.g., an anticoagulant, a vasoconstrictor, or a vasodilator. Thus, the guiding member may not only cause a mechanical expansion of the tissue or vessel, but may also influence the physiological condition. In this way, the introduction of a vessel expansion system may also be further improved or facilitated due to a pharmacologically induced physiological reaction of the patient, so that tissue damage may be avoided as far as possible.

A surface of the body may further include an adhesive coating to bond or attach the body to the controllable member. For example, a coupling with an internally located controllable member may be reinforced by an adhesive coating on the inner surface of the guiding member so that the guiding member is adjacent to the controllable member even during the insertion of the vessel expansion system and relative movement may be prevented accordingly without further mechanical connection. An outer surface may also be provided with such an adhesive coating, if the guiding member is surrounded by the controllable member in the coupled state.

As described above, the body of the guiding member, e.g., does not to include any wrinkles, regardless of its radial extension, in order to avoid a possible impairment of the tissue, to facilitate insertion and to control the diameter. Accordingly, the body can include a homogeneous and continuous circumference. The circumference is, e.g., uniform and continuous from a proximal region to a distal region, so that an orientation or alignment of the guiding member has essentially the same effect for each radial orientation.

To further facilitate the insertion of a vessel expansion system and the rotation and tilt of the vessel expansion system, for example during the insertion and control or navigation of the vessel expansion system, and to adapt the shape of the guiding member as closely as possible to a vascular structure of the patient, the circumference of the body can have a substantially circular or ellipsoidal shape in cross-section. Thus, the guiding member may have an essentially cylindrical shape or be designed as a tube. This shape may also be adapted to the internal shape of a cannula or catheter, making it easier to insert such a cannula over the guiding member.

Independent of the arrangement of the guiding member with respect to the controllable member, the body may further include a distal tip which can be formed as an atraumatic and/or rounded tip.

Because the body extends entirely along the controllable member, the body may include a guide surface at the distal region, both in the embodiment as the core of a vessel expansion system and in the alternative embodiment as the outer sleeve or sheath of the vessel expansion system. By means of the tip, a structure may be provided in the assembled or coupled state of the vessel expansion system, which may initially be inserted into the tissue after a corresponding tissue incision and which, for example, is conical or pyramidal in shape in order to insert the distal region into the tissue. If the guiding member is configured as an outer sleeve or sheath, the tip also seals the distal region of the body in a fluid-tight manner so that no fluids may reach the inner controllable member via the distal region.

The tip can be configured in such a way that it may be deformed upon radial expansion of the body. When a change is made to the controllable member, which adjusts the radial extent and an expansion is caused, the deformation of the tip may ensure that no material stress is caused and that the expansion can extend evenly towards the distal region. This not only improves structural stability, but also prevents a cannula from not or not sufficiently being guided over the vessel expansion system due to irregularities in the circumference of the body.

The body may also be formed of several material layers, e.g., two or more material layers, wherein an inner material layer has a greater stiffness than an outer material layer. Accordingly, sufficient strength or rigidity of the guiding member is provided. A softer outer layer may also prevent accidental damage to the tissue during insertion. The inner material layer can include a polymer, e.g., polyurethane, and the outer material layer can include silicone or silicone-like material.

To couple the guiding member to a controllable member, the body can include a connecting section for coupling the body to a corresponding connecting section of the controllable member. Because the body and a surface of the controllable member can adjoin each other, there is already a corresponding contact surface over the entire extension of the guiding member. For example, a connecting section at the distal and/or at the proximal region hence provides a sufficient connection or restricts or even prevents relative movement in axial direction, radial direction, and/or direction of rotation. In this way, for example, the guiding member may be guided or pushed onto the controllable member, wherein a connection is automatically achieved when the element is fully inserted.

For connecting, the connecting section may include, for example, one or more projections or protrusions, bulges, latching hooks, recesses, grooves, undercuts, and/or a thread. For example, one or more protrusions may be provided which, in the coupled state, are received by recesses in the corresponding region of the controllable member and which prevent relative movement, at least in the axial direction and in a direction of rotation. For example, undercuts in the distal region may be used to lock the device in place, or a groove may be provided on the guiding member which is engaged by a handle of a vessel expansion system with a locking hook in the coupled state so that axial movement is prevented and the guiding member and the coupled controllable member are biased by the handle. Alternatively, one or more barbs or a fir tree connector may be used to accommodate different diameters.

In some embodiments, the connection section is adapted to achieve a press fit, a positive fit, or a snap fit with the controllable member. This allows a sufficient connection to be provided, while at the same time providing haptic feedback during the coupling indicating a correct connection. However, the connection may still be released, either by moving in a predefined direction or by using an appropriate tool, so that the guiding member may be separated from the controllable member after use.

Alternatively, or in addition, it may be provided that the guiding member in the proximal region includes one or more magnetic elements for establishing a magnetic coupling with the controllable member. For example, the guiding member and the controllable member may be provided with a respective magnetic element, wherein the magnetic elements are polarized differently. Accordingly, haptic feedback may be provided after a correct connection and the connection may still be easily released. This allows the connection section to be provided without the need for complicated technical design, especially as no special shaping is required, which also significantly simplifies fabrication and further improves the structural stability of the guiding member.

To temporarily attach a cannula to the guiding member and/or to ensure that a cannula has been fully inserted, the body may also include one or more flexible bulges at the proximal and/or distal regions for coupling a cannula. For example, a projection or protrusion may be provided at the distal portion of the guiding member over the entire circumference in a direction of rotation, which extends radially outwards so that a cannula is limited in the axial direction when it is inserted. This prevents the cannula from being guided beyond the desired position, i.e., the cannula may be inserted and positioned correctly.

As described above, the guiding member may be used as an adapter, which may serve either as an outer sheath or as the core of a vessel expansion system. The guiding member can be configured as a single-use item or disposable. This has the particular advantage that, if the guiding member is configured as a sleeve or sheath, the controllable member to be coupled may be sealed in a fluid-tight manner and therefore only the guiding member comes into contact with fluids such as the patient's blood. While the controllable member, which may include various components, may be used for further applications, the guiding member may be disposed of as a disposable item and replaced with a new (unused) guiding member for further applications of the system so that hygiene standards may be met.

The above aspect is further achieved by a controllable vessel expansion system for insertion into an anatomical region of a patient. The vessel expansion system includes a guiding member as described above and a controllable member, wherein the guiding member and the controllable member are releasably coupled to each other.

In some embodiments, the controllable member includes a fluid chamber extending from a proximal region to a distal region of the vessel expansion system along the longitudinal axis of the vessel expansion system, and wherein an amount of fluid in the fluid chamber defines a radial extent of the fluid chamber and a radial extension of the vessel expansion system.

The fluid chamber can be adapted to the shape of the guiding member, so that the fluid chamber and the body of the guiding member are adjacent to each other. The guiding member may either be surrounded by the fluid chamber or may surround the fluid chamber, wherein the guiding member is coupled to the controllable member as an adapter.

The variable radial extension of the vessel expansion system allows that the vessel expansion system is initially inserted into a patient's tissue and that the diameter of the vessel expansion system is subsequently increased by adjusting the amount of fluid so that the tissue may expand. Accordingly, the vessel expansion system may function as an independent unit so that a guide wire or the use of the “Seldinger technique” may be omitted. The fluid chamber may be adapted for a specific fluid volume so that the fluid chamber may be filled with a fluid within a specified volume range.

The diameter of the vessel expansion system may thus be increased to the diameter required for the medical application by adjusting or increasing the amount of fluid in the fluid chamber without the need for additional dilators.

However, the guiding member is not deformed during expansion, so that the circumference is essentially homogeneous, even if the radial extension changes. The vessel expansion system is also sufficiently stable in shape, such that a buckling also during use is not to be expected.

In some embodiments, the controllable member of the vessel expansion system is made from a rigid but elastically deformable biocompatible material. Typically, a tear-resistant material is used, e.g., a plastic material, for example selected from the group of polyurethane, PVC, or silicone. The controllable member can form a fluid chamber, which is configured for holding a fluid. The controllable member may thus be configured, for example, as a cuff-shaped or sleeve-like structure, which may surround the guiding member like a coating or jacket, which has an inner and an outer wall, between which a cavity is positioned for establishing a chamber-like configuration. Thus, the controllable member does typically not form a simple shell or envelope structure, such as a type of a sheath, but can be characterized as a fluid chamber with an inner and an outer wall, which is formed by a cavity for holding, e.g., a fluid. The controllable member can have a wall thickness of the inner and the outer wall, respectively, in the range of 0.3 to 5 mm, 0.5 mm to 3 mm, or 0.5 to 2 mm.

The controllable member itself or the vessel expansion system can have no balloon-like structure.

The vessel expansion system can have no guide wire.

In some embodiments, the controllable member, which has, e.g., the afore-mentioned characteristics, surrounds the guiding member in circumferential direction. In some embodiments, the guiding member surrounds the controllable member, which is arranged in the interior and which has the above characteristics.

Between the controllable member and the guiding member, an intermediate member, e.g., of layer-like or layered envelope or sleeve-like configuration, may be provided by the vessel expansion system. The intermediate member may have the function of a sliding layer. Hereby, the sliding behavior of the controllable member towards the guiding member or vice versa may be improved. The sliding layer may be formed by a low friction material or may be coated, e.g., on both surfaces, with such a material, typically by a gliding-eager and abrasion resistant plastic material, e.g., silicone-polyester (PES) or fluor polymers, e.g. by PTFE. Alternatively or additionally, a coating with, e.g., a gel-like lubricant, e.g., based on silicone (e.g. polysiloxane or modified polysiloxane) or based on glycerine, may be provided. A coating with a, e.g., hydrophilic lubricant, may be provided on one or both surfaces of the intermediate member forming a sliding layer.

Different fluids or liquids may be used to fill the fluid chamber, for example biocompatible saline solutions or a dilatant fluid. Accordingly, the physical state of the fluid may be varied by shear forces.

This allows the vessel expansion system to be used as a rigid structure for inserting the vessel expansion system into a tissue incision and then as a flexible structure during guidance or navigation in a patient's blood vessel, depending on the shear forces acting on the fluid. Also, a certain shape or a given state may be frozen, so to speak, which can, however, be reversed by adjusting the shear forces accordingly. The physical state of the fluid is therefore completely reversible.

Furthermore, it may be provided that the fluid chamber at the distal region includes a movable region, which can be movable by the action of shear forces on the fluid. For example, a pre-bend defined by an anatomical structure may be frozen or a shape may be manually defined in the movable region, wherein this shape may be maintained by applying a corresponding shear force, but may also be released again, when the shear force is adjusted, so that the vessel expansion system is moved back to its original state or a shape corresponding to the anatomy. The movable region thus allows adaptation to a specific anatomical geometry and an improved control or facilitating of navigation or guidance of the vessel expansion system in the patient, e.g., into more complex anatomical structures.

For example, the fluid chamber may be filled using a flexible reservoir, so that, for example, a predefined volume may be filled into the fluid chamber in order to achieve a corresponding radial extension of the vessel expansion system. However, the vessel expansion system can include a pumping device for introducing and discharging fluid into the fluid chamber and a steering device for controlling the controllable member at the distal portion of the vessel expansion system. This provides a more versatile application of the vessel expansion system and also allows improved adaptation of the diameter to the anatomical structure.

To ensure that the vessel expansion system is correctly inserted and the distal region is positioned in place, the vessel expansion system may also include a sensor element at the distal region to determine the position of the distal end of the vessel expansion system. The sensor element may, for example, be integrated in the guiding member, for example also in the region of a tip. This makes it possible, for example, to monitor whether the vessel expansion system is located in the region of the right atrium, so that a cannula may then be inserted over the vessel expansion system to relieve the right heart. It is to be understood that such a monitoring may also be performed during the introduction of the vessel expansion system to assist insertion or control or navigation, hence further facilitating the corresponding medical application.

The sensor element can include a magnetic field sensor, an ultrasonic sensor, or an inductive position sensor. For example, a magnetic field sensor may be provided in the distal region of the guiding member, wherein an absolute position may be determined by means of a reference image using CT or MRT.

Alternatively, or additionally, the sensor element may be configured as an electrically conductive material in the guiding member. For example, the guiding member may be an integrated wire reinforcement, which is integrated as an electrically conductive material and is spirally arranged in the longitudinal direction of the guiding member and electrically isolated therein. The wire may, for example, form an electrical connection at the proximal region of the vessel expansion system, where an electrical potential is received with a cable, which may be passed on to a measuring device for a capacity measurement. The capacity measurement serves as a measure for the extension of the vessel expansion system or the guiding member, so that a position of the vessel expansion system, for example within a blood vessel, may be determined accordingly by software and optionally with a visualization in a reference image. Furthermore, inductive position sensors may also be used, which make it possible to determine a position by applying a magnetic field.

Accordingly, the sensor element may be coupled with a monitoring system for outputting a warning signal in the event of a deviation from a predefined position.

Further sensors may also be provided or integrated in the vessel expansion system, which provide relevant information to the patient in the region of the vessel expansion system. The vessel expansion system can include at least one sensor unit for determining a physiological parameter of the patient, e.g., for determining the blood pressure and/or the blood flow of the patient. This enables the operating personnel of the vessel expansion system to obtain information about a pathophysiological condition directly at the relevant anatomical site, for example when dilating or expanding a blood vessel, by means of appropriate measurements, so that, for example, a therapy or a planned medical intervention may be adapted to the patient's needs as early as possible in situ. Further sensor technologies, e.g., a pH sensor or an O₂ partial pressure sensor, may be provided.

In some embodiments, the vessel expansion system is configured for use in expanding a region of a blood vessel of a patient.

As described above, the guiding member may be configured as an adapter and may accordingly surround a controllable member. This allows the controllable member to be fluidly sealed and prevents potential contamination of the controllable member so that it may be used for further applications. The controllable member, which may include other components in addition to the fluid chamber, is thus protected against contamination, wherein the guiding member may be disposed of as an adapter in the form of a disposable item after use, while the device may continue to be used. The controllable member can be made of a durable material, e.g., metal, which allows easy steam sterilization before reuse. Accordingly, the guiding member can define an inner cavity, wherein the controllable member is arranged in the inner cavity. Alternatively, it may be provided that the guiding member defines an inner core of the vessel expansion system and is surrounded circumferentially by the controllable member.

In some embodiments, the controllable vessel expansion system described herein does not require a guide wire.

Further, a catheter is suggested having an inner cavity extending from a proximal region of the catheter to a distal region of the catheter, wherein the catheter includes a vessel expansion system as described in the above and disposed within the inner cavity of the catheter. Accordingly, the catheter or alternatively also a cannula may be provided as a system, wherein the catheter can be configured in such a way that it may be inserted or pushed onto the vessel expansion system after insertion of the vessel expansion system.

The catheter described herein does not have a guide wire.

The above aspect is further achieved by a method to expand an anatomical region of a patient. The procedure includes at least the following:
providing a vessel expansion system as described above;
introducing the vessel expansion system into the anatomical region of the patient in a state in which the vessel expansion system includes a minimum radial extension; and
increasing the amount of fluid in the fluid chamber to increase the radial extent and circumference of the vessel expansion system.

Accordingly, due to its variable radial extension, the vessel expansion system may easily be inserted with the smallest possible diameter into the tissue or blood vessel and the diameter of the vessel expansion system may then be increased by adjusting the amount of fluid so that the tissue may be expanded. The insertion of a guide wire and the successive insertion of additional dilators with different diameters may thus be completely dispensed with, thereby making the performance of the method significantly easier, faster and with a lower risk for the patient.

The vessel expansion system can also be configured in such a way that, after the anatomical region has expanded, a cannula is inserted or guided onto the vessel expansion system and wherein the insertion is monitored by a navigation system coupled to the vessel expansion system. For example, a distal region of the vessel expansion system may be configured to couple to a distal region of a cannula and may be equipped with a sensor element in this region so that the positioning of the cannula may be checked. It may also be provided that a distal region of the cannula is provided with a sensor element so that the determination of the position of the cannula and a coupled vessel expansion system is only possible after insertion of the cannula. This allows the guiding member and the vessel expansion system to be more compact.

Accordingly, a method for monitoring the positioning of a cannula is suggested, wherein a cannula is inserted on a vessel expansion system as described in the above and wherein the position of a distal end of the vessel expansion system is monitored by a monitoring system coupled to the vessel expansion system. Thus, the position of the cannula may be monitored. Also, as described above, the guiding member may be configured to form a “core” of the vessel expansion system and to be surrounded by the controllable member. In such an embodiment the vessel expansion system may also be configured directly as a cannula on the outer circumference, thereby further simplifying the monitoring of the position of the cannula during the insertion of the vessel expansion system.

### BRIEF DESCRIPTION OF THE DRAWINGS

Further embodiments according to the invention are explained in more detail in the following description of the Figures, showing:

FIG. 1 is a schematic representation of a vessel expansion system with the guiding member in an embodiment;

FIG. 2 is a schematic representation of the vessel expansion system as shown in FIG. 1 with a second radial extension;

FIGS. 3A and 3B show a schematic detail view in a longitudinal section of a guiding member with a coupled controllable member at different radial extensions;

FIGS. 4A and 4B schematically show different configurations of a flexible section in a cross-section of a guiding member with a coupled controllable member;

FIGS. 5A and 5B show a schematic detail view of the tip region at different radial extensions; and

FIG. 6 is a schematic representation of a vessel expansion system with the guiding member according to a further embodiment.

### DETAILED DESCRIPTION

In the following, embodiments will be explained in more detail with reference to the accompanying figures. In the Figures, corresponding, similar, or like elements are denoted by identical reference numerals and repeated description thereof may be omitted in order to avoid redundancies.

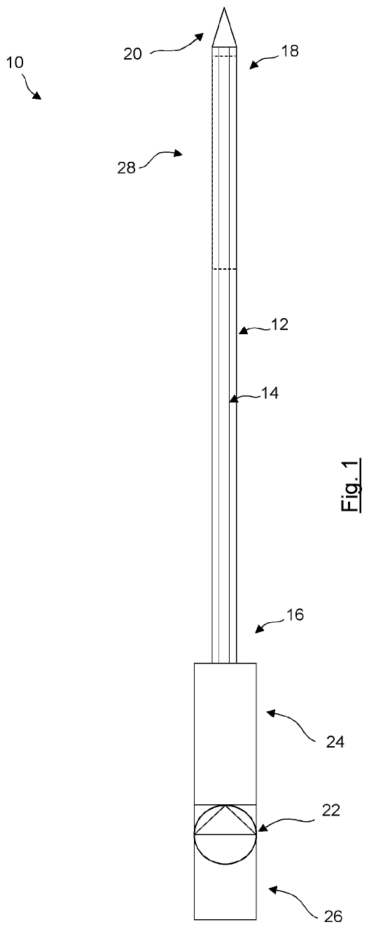

FIG. 1 schematically shows a controllable vessel expansion system 10, wherein a guiding member 12 and a controllable member 14 are coupled to each other. In this embodiment, the guiding member 12 is configured as a sleeve or adapter so that the guiding member 12 surrounds the controllable member 14 arranged inside. The guiding member 12 is formed substantially as an elongate body extending along a longitudinal axis of the vessel expansion system 10 from a proximal region 16 to a distal region 18 with an inner surface of the guiding member 12 being adjacent to an outer surface of the controllable member 14, such that from the proximal region 16 to the distal region 18 it is fluidically sealed by the guiding member 12.

Furthermore, a tip is provided at the distal region 18 of the guiding member 12, which may be formed in one piece with the guiding member 12. As a result, no liquids may penetrate into the interior or into the controllable member 14 via the distal region 18. Thus, the guiding member 12 may be used as an adapter for the controllable member 14 and may be adapted, for example, as a disposable item while the controllable member 14 is protected against contamination and may be reused.

The body of the guiding member 12 is also shown in FIG. 1 at a first radial extension, with the vessel expansion system 10 having a minimum diameter. However, the radial extension may be varied by the controllable member 14, which is essentially formed as a fluid chamber. For example, the amount of fluid in the fluid chamber may determine a radial extension of the vessel expansion system 10, whereby the amount of fluid may be adjusted by the pumping device 22, which is integrated in a handle 26, for example as a micro-pump. When the fluid volume changes, the volume of the fluid chamber also changes, so that the guiding member 12 is pushed outwards. The body of the guiding member 12 is optionally made of an elastic material and may be deformed accordingly by the flexible outer wall, so that the radial extension of the vessel expansion system 10 is adapted accordingly.

The guiding member 12 and the controllable member 14 are coupled together at each radial extension, which may be further reinforced by an optional connecting section (not shown) and/or an adhesive coating of the adjacent surfaces. Also, the expansion or radial extension does not produce any wrinkles or deformations on the outer surface of the guiding member 12, so that the body continues to have a homogeneous and continuous circumference.

The vessel expansion system 10 also includes a steering device 24, which is configured to control the movement of the controllable member 14 at the distal region 18 or at the tip 20. The vessel expansion system 10 as well as the guiding member 12 and the controllable member 14 have a movable region 28 at the distal region 18, which is shown schematically with the dotted lines. The steering device 24 hence belongs, like the pump device 22 and the handle 26, to the outer structure of the vessel expansion system 10.

The steering device 24 and the movable region 28 allow the distal region 18 of the vessel expansion system 10 to be rotated or steered so that the tip 20 of the vessel expansion system 10 may be variably aligned and oriented. For example, a minimum bending radius of about 15 to 25 mm may be provided and this may be increased to 40 to 60 mm or up to about 100 mm, with the vessel expansion system 10 being able to be bent up to about 180° accordingly, so that the vessel expansion system 10 may be adapted to larger and/or more complex anatomical structures. Accordingly, a minimum diameter of the vessel expansion system 10 may also include 12 Fr. or less, with the upper limit of the diameter being approximately 32 Fr., for example, so that the individual vessel expansion system 10 allows continuous expansion between approximately 12 Fr. or less and approximately 32 Fr. Furthermore, different lengths of the vessel expansion system 10 may be selected, for example between about 100 and 700 mm, e.g., between about 140 and about 650 mm.

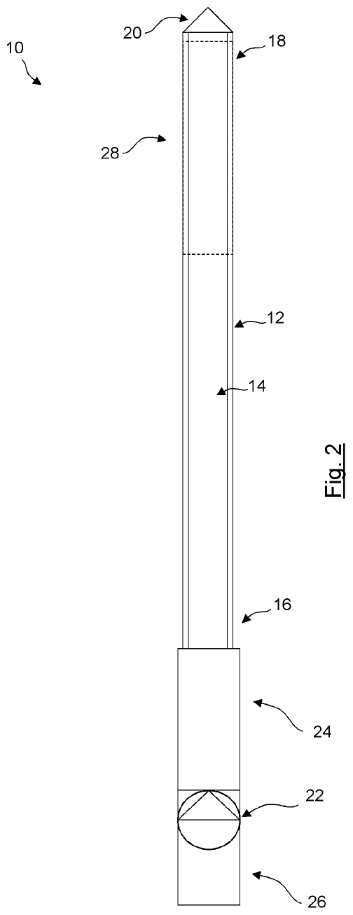

A second radial extension of the vessel expansion system 10 is shown in FIG. 2, wherein the amount of fluid in the fluid chamber of the controllable member 14 has been increased accordingly. Since the guiding member 12 and the controllable member 14 adjoin each other it is also evident that the diameter is defined and predetermined correspondingly also upon a change in the radial extension of the controllable member 14 and the guiding member 12.

It may also be seen here that the larger volume of the fluid chamber not only causes an expansion of the vessel expansion system 10, but also, due to the elastic material, an expansion of the guiding member 12. This also makes the outer wall thinner compared to the first radial extension. However, the outer surface of the guiding member 12 retains its homogeneous and continuous configuration so that wrinkles may be avoided during expansion.

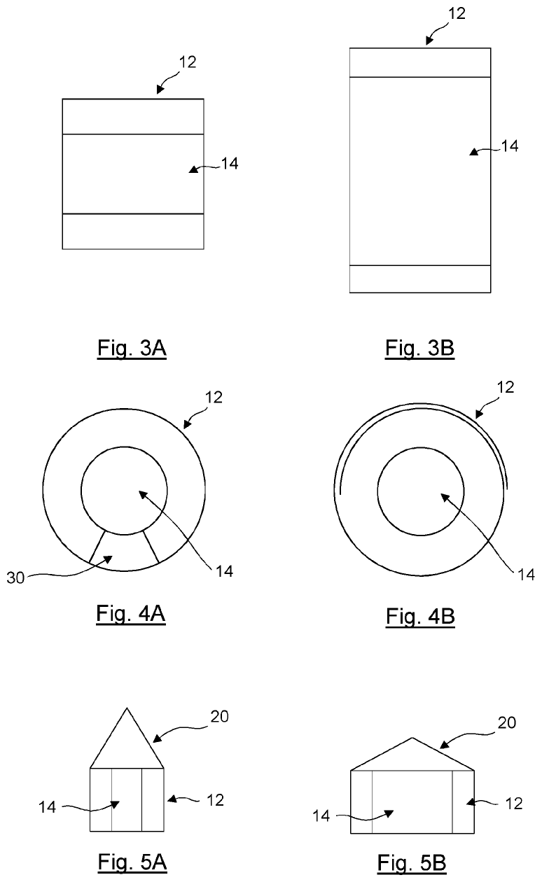

The different radial extension and the effect on the guiding member 12 is shown in a schematic detail view in FIGS. 3A and 3B.

Accordingly, an amount of fluid in the controllable member 14 or in the fluid chamber as shown in FIG. 3A may cause a first radial extension and diameter of the vessel expansion system, wherein an increase in the amount of fluid, for example by the pumping means of the vessel expansion system, causes a corresponding change in volume of the fluid chamber so that a second radial extension is achieved at a predefined diameter of the vessel expansion system, as shown in FIG. 3B. The thickness of the guiding member 12 changes only slightly in proportion.

The fluid that may be used for the vessel expansion system may basically be any liquid, e.g., a biocompatible liquid such as a salt solution. However, a dilatant fluid, such as a Casson fluid or a Bingham fluid, can also be used. This allows the rigidity of the vessel expansion system to be affected by shear forces, allowing the fluid to vary between a liquid state and a near solid state. In this way, a form of the vessel expansion system may be specified, for example actively by deformation of the movable region or passively by anatomical structures, and reversibly fixed or frozen in this form. Thus, for example, a rigid and, if necessary, adapted shape at the initial insertion of the vessel expansion system may facilitate insertion into a tissue section and, subsequently, a flexible vessel expansion system may be advantageous in guiding and navigating a blood vessel.

The guiding member may also have a flexible section 30 as an alternative or in addition to the elastic configuration, as shown in FIGS. 4A and 4B in a cross-section of a guiding member 12 with a coupled controllable member 14.

It may be seen from these Figures that the circumference of the body of the guiding member 12 in cross-section has an essentially circular or ellipsoidal shape, which can be used, for example, for the expansion of tissue and blood vessels.

As shown in FIG. 4A, the flexible section 30 may be provided at a section of the body in the circumferential direction and may also extend along the longitudinal axis of the vessel expansion system accordingly. For example, section 30 may be formed from an elastic material such as an elastomer which is stretchable and is deformed as the amount of fluid changes, thereby increasing the circumference of the guiding member 12 and achieving a predefined diameter of the vessel expansion system. However, the remaining part or body of the guiding member is not expanded or only slightly expanded, depending on the material used, but may be deformed to maintain the original shape of the vessel expansion system.

An alternative configuration of the flexible section 30 is shown in FIG. 4B, wherein the body is formed in cross-section as a spiral shape and wherein a degree of unfolding of the spiral shape defines a radial extension of the body. Accordingly, a change in the amount of fluid in the controllable member 14 or in the fluid chamber may cause the guiding member 12 to expand in the radial direction, wherein the guiding member rolls out or unfolds accordingly. The material may also be elastic, so that the guiding member 12 also retracts when the fluid quantity is reduced and the expansion is correspondingly reversible.

As already shown in FIG. 2 with respect to FIG. 1, the shape of the tip 20 may also be adapted to different diameters of the vessel expansion system, as shown in FIGS. 5A and 5B in a schematic detail view of the tip region.

The tip 20 is formed in such a way that it may be deformed by radial expansion of the body. It may also be made of an elastic material. Due to the radial extension, the tip is also extended in a radial direction, wherein the length of the tip 20 is shortened accordingly.

Although the Figures show that the fluid chamber of the controllable member 14 does not protrude into the tip 20, this may be optionally provided by shaping the tip 20 and the controllable member 14 accordingly, so that a change in the fluid quantity may act directly on the tip 20 and the tip 20 may also be dimensioned smaller and/or variably. Furthermore, the tip 20 may be rounded and can be formed as an atraumatic tip 20.

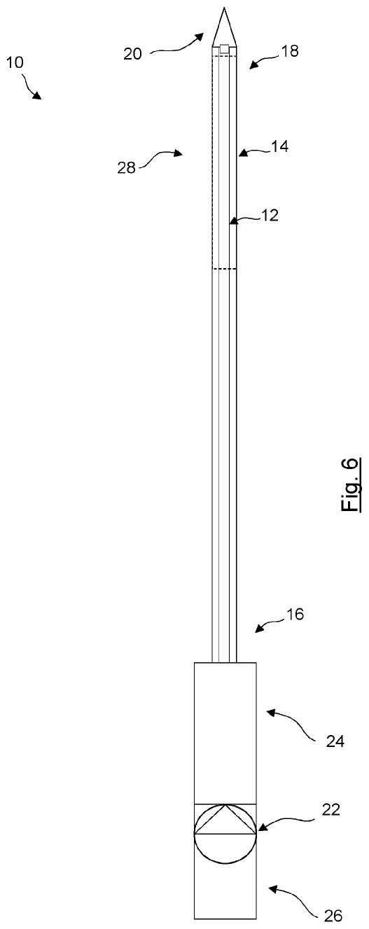

An alternative embodiment of the vessel expansion system 10 is shown in FIG. 6 in a schematic representation with a guiding member 12 and a coupled controllable member 14.

The features of the vessel expansion system 10 here are essentially identical to the features shown in FIG. 1. In this embodiment, however, the guiding member 12 is designed as the core of the vessel expansion system 10 and is surrounded by the controllable member 14. Accordingly, the fluid chamber is arranged circumferentially. When the fluid quantity is increased, the radial extension of the fluid chamber also increases it. The radial extension of the guiding member 12 does not change or changes only slightly.

In this configuration, the guiding member 12 may also be used as an adapter, whereby the guiding member 12 provides structural stability for the vessel expansion system 10 and prevents the controllable member 14 from protruding into the interior of the vessel expansion system when the fluid quantity changes.

Furthermore, such a configuration may be integrated into a cannula so that a cannula with a controllable member 14 may include an integrated movement controller and fluid chamber and the guiding member 12 may be inserted for use with the vessel expansion system 10 or into an inner cavity of the cannula.

Where applicable, all the individual features depicted in the exemplary embodiments may be combined and/or exchanged without leaving the scope of the invention.

### LIST OF REFERENCE NUMERALS

10 Vessel expansion system
12 Guiding member
14 Controllable member
16 Proximal region
18 Distal region
20 Tip
22 Pumping device
24 Steering device
26 Handle
28 Moving region
30 Flexible section

1-47. (canceled)

48. A guiding member for a controllable vessel expansion system, the guiding member comprising:
an elongate body couplable to a controllable member of the vessel expansion system, the elongate body being aligned along a longitudinal axis of the vessel expansion system from a proximal region to a distal region of the vessel expansion system in a coupled state, wherein the elongate body is expandable in a radial direction, and wherein the elongate body is releasably couplable to the controllable member, which together with the guiding member defines a radial extension of the vessel expansion system.

49. The guiding member according to claim 48, wherein the elongate body is adapted to be circumferentially surrounded by the controllable member in the coupled state.

50. The guiding member according to claim 48, wherein the elongate body is a sleeve or sheath and is adapted to completely and fluidly-tight enclose the controllable member in the coupled state.

51. The guiding member according to claim 50, wherein the elongate body is movable between a first radial extension, wherein the vessel expansion system comprises a minimum diameter, and a second radial extension, wherein the elongate body defines a predefined diameter of the vessel expansion system in each radial extension.

52. The guiding member according to claim 50, wherein the elongate body is continuously expandable in a radial direction.

53. The guiding member according to claim 50, wherein the elongate body comprises a flexible section in a circumferential direction which defines a radial extension of the body.

54. The guiding member according to claim 53, wherein the elongate body is formed of an elastic material.

55. The guiding member according to claim 50, wherein the elongate body has a spiral shape in cross-section and wherein a degree of unfolding of the spiral shape defines a radial extension of the elongate body.

56. The guiding member according to claim 50, wherein an outer surface of the elongate body comprises a friction-reducing coating.

57. The guiding member according to claim 50, wherein an outer surface of the elongate body is adapted to discharge fluid and comprises grooves, lamellae, and/or a honeycomb structure.

58. The guiding member according to claim 50, wherein an outer surface of the elongate body is coated with a medicament, the medicament being a coagulation inhibitor, a vasoconstrictor, or a vasodilator.

59. The guiding member according to claim 49, wherein a surface of the elongate body comprises an adhesive coating for bonding the elongate body to the controllable member.

60. The guiding member according to claim 48, wherein the elongate body comprises a homogeneous and continuous circumference.

61. The guiding member according to claim 48, wherein the circumference of the elongate body comprises a substantially circular or ellipsoidal shape in cross-section.

62. The guiding member according to claim 48, wherein the elongate body comprises an atraumatic and/or rounded tip arranged at the distal region of the vessel expansion system

63. The guiding member according to claim 62, wherein the tip is configured to deform upon radial expansion of the elongate body.

64. The guiding member according to claim 48, wherein the elongate body is formed of a plurality of material layers, wherein an inner material layer has a greater stiffness than an outer material layer.

65. The guiding member according to claim 64, wherein the inner material layer comprises polyurethane and the outer material layer comprises silicone.

66. The guiding member according to claim 48, wherein the elongate body comprises a connecting portion for coupling the elongate body to a corresponding connecting portion of the controllable member.

67. The guiding member according to claim 66, wherein the connecting portion comprises one or more protrusions, bulges, latching hooks, recesses, grooves, undercuts, and/or threads.

68. The guiding member according to claim 66, wherein the connecting portion is configured for providing a press fit, a positive fit, or a snap fit with the controllable member.

69. The guiding member according to claim 66, wherein the guiding member comprises one or more magnetic elements in the proximal region of the vessel expansion system for providing a magnetic coupling with the controllable member.

70. The guiding member according to claim 48, wherein the elongate body comprises one or more flexible bulges at the proximal region and/or the distal region of the vessel expansion system for coupling a cannula.

71. The guiding member according to claim 48, wherein the guiding member is a single-use item or disposable.

72. A controllable vessel expansion system for insertion into an anatomical region of a patient, the controllable vessel expansion system comprising:
a controllable member, and
a guiding member comprising an elongate body couplable to the controllable member, the elongate body being aligned along a longitudinal axis of the vessel expansion system from a proximal region to a distal region of the vessel expansion system in a coupled state, wherein the elongate body is expandable in a radial direction, and wherein the elongate body is releasably couplable to the controllable member, which together with the guiding member defines a radial extension of the vessel expansion system,
wherein the guiding member and the controllable member are releasably coupled together.

73. The controllable vessel expansion system according to claim 72, wherein the controllable member comprises a fluid chamber extending from the proximal region to the distal region of the vessel expansion system along the longitudinal axis of the vessel expansion system, wherein an amount of fluid in the fluid chamber defines a radial extension of the fluid chamber and the radial extension of the vessel expansion system.

74. The controllable vessel expansion system according to claim 73, wherein a physical state of the fluid is variable by shear forces.

75. The controllable vessel expansion system according to claim 73, wherein the fluid chamber comprises a movable region at the distal region of the vessel expansion system, wherein the movable region is movable based on actuated shear forces on the fluid.

76. The controllable vessel expansion system according to claim 73, further comprising:
a pump device for introducing and discharging fluid into the fluid chamber; and
a steering device for controlling the controllable member at the distal portion of the vessel expansion system.

77. The controllable vessel expansion system according to claim 72, further comprising a sensor element at the distal region of the vessel expansion system for determining a position of a distal end of the vessel expansion system.

78. The controllable vessel expansion system according to claim 77, wherein the sensor element is formed as an electrically conductive material in the guiding member as an integrated wire reinforcement, or wherein the sensor element comprises a magnetic field sensor, an ultrasonic sensor, or an inductive position sensor.

79. The controllable vessel expansion system according to claim 77, wherein the sensor element is couplable to a monitoring system for outputting a warning signal in case of a deviation of the position determined by the sensor element from a predetermined position.

80. The controllable vessel expansion system according to claim 72, further comprising at least one sensor unit for determining a blood pressure and/or a blood flow of the patient.

81. The controllable vessel expansion system according to claim 72, wherein the guiding member defines an inner cavity and wherein the controllable member is disposed within the inner cavity or wherein the guiding member defines an inner core of the vessel expansion system and is circumferentially surrounded by the controllable member.

82. The controllable vessel expansion system according to claim 81, wherein the controllable member is made from a plastic material.

83. The controllable vessel expansion system according to claim 72, wherein the guiding member defines an inner core of the vessel expansion system and is circumferentially surrounded by the controllable member, wherein the controllable member is a fluid chamber forming a cavity for holding a fluid and is made from a rigid but elastically deformable material.

84. The controllable vessel expansion system according to claim 83, wherein the controllable vessel expansion system does not include a balloon-like structure and a guide wire, and wherein the controllable member forming a fluid chamber has a cuff-shaped or sleeve-like configuration with an inner and an outer wall, the outer wall surrounding the cavity.

85. The controllable vessel expansion system according to claim 72, wherein the controllable vessel expansion system does not include a balloon-like structure.

86. The controllable vessel expansion system according to claim 72, wherein the controllable member has a cuff-shaped or sleeve-like configuration.

87. The controllable vessel expansion system according to claim 72, wherein the controllable vessel expansion system does not include a guide wire.

88. A catheter having an inner cavity extending from a proximal region of the catheter to a distal region of the catheter, the catheter comprising a vessel expansion system according to claim 72 which is disposed within the inner cavity of the catheter.

89. The catheter with an inner cavity according to claim 88, wherein the catheter does not have a guide wire.

90. A method of expanding an anatomical region of a patient, the method comprising:
providing a controllable vessel expansion system according to claim 72;
introducing the vessel expansion system into the anatomical region of the patient in a state wherein the vessel expansion system comprises a minimum radial extension; and
increasing the amount of fluid in the fluid chamber to increase the radial extension and circumference of the vessel expansion system.

91. The method according to claim 90, wherein after expansion of the anatomical region a cannula is inserted onto the vessel expansion system and wherein the insertion is monitored by a navigation system coupled to the vessel expansion system.

92. A method of monitoring a positioning of a cannula, the method comprising:
inserting a cannula onto a vessel expansion system, the vessel expansion system comprising:
a controllable member comprising a fluid chamber extending from a proximal region to a distal region of the vessel expansion system along a longitudinal axis of the vessel expansion system, wherein an amount of fluid in the fluid chamber defines a radial extension of the fluid chamber and a radial extension of the vessel expansion system, and
a guiding member comprising an elongate body couplable to the controllable member, the elongate body being aligned along the longitudinal axis of the vessel expansion system from the proximal region to the distal region of the vessel expansion system in a coupled state, wherein the elongate body is expandable in a radial direction, and wherein the elongate body is releasably couplable to the controllable member, which together with the guiding member defines the radial extension of the vessel expansion system,
wherein the guiding member and the controllable member are releasably coupled together,
wherein the position of the distal end of the vessel expansion system is monitored by a monitoring system coupled to the vessel expansion system.
